(19) United States
(12) Patent Application Publication
(10) Pub. No.: US 2025/0232444 A1
(43) Pub. Date: Jul. 17, 2025

(54) METHOD AND APPARATUS FOR PERFORMING IMAGE ENHANCEMENT USING A NEURAL NETWORK IN A MEDICAL IMAGING SYSTEM

(71) Applicants: THE REGENTS OF THE UNIVERSITY OF CALIFORNIA, Oakland, CA (US); CANON MEDICAL SYSTEMS CORPORATION, Tochigi (JP)

(72) Inventors: Yu-Jung TSAI, Vernon Hills, IL (US); Wenyuan QI, Vernon Hills, IL (US); Jinyi QI, Oakland, CA (US); Li YANG, Vernon Hills, IL (US); Evren ASMA, Vernon Hills, IL (US); Jeffrey KOLTHAMMER, Vernon Hills, IL (US)

(73) Assignees: THE REGENTS OF THE UNIVERSITY OF CALIFORNIA, Oakland, CA (US); CANON MEDICAL SYSTEMS CORPORATION, Tochigi (JP)

(21) Appl. No.: 19/016,764

(22) Filed: Jan. 10, 2025

Related U.S. Application Data
(60) Provisional application No. 63/620,711, filed on Jan. 12, 2024.

(51) Int. Cl. G06T 7/00; G06T 5/50; G06T 5/60; G06T 7/11; G06T 7/174; G06T 11/00; G06V 10/26; G06V 10/774; G06V 10/82; G16H 70/60

(52) U.S. Cl. CPC G06T 7/0014; G06T 5/50; G06T 5/60; G06T 7/11; G06T 7/174; G06T 11/005; G06T 11/006; G06T 11/008; G06V 10/273; G06V 10/774; G06V 10/82; G16H 70/60; G06T 2207/10104; G06T 2207/20021; G06T 2207/20081; G06T 2207/20084; G06T 2207/20132; G06T 2210/22; G06T 2210/41; G06T 2211/441; G06T 2211/448

(57) ABSTRACT

A method for performing image enhancement using a neural network is provided. The method includes acquiring an image of an imaging object, and applying the acquired image to a trained neural network to generate an image-enhanced image of the imaging object. The neural network was trained by receiving a training image pair including a first image and a second image, performing an image intensity preprocessing on the received image pair to generate a preprocessed first image and a preprocessed second image, such that the preprocessed first image has a first intensity range covering a portion of an intensity range of the first image, and the preprocessed second image has a second intensity range covering a portion of an intensity range of the second image, and training the neural network using the preprocessed first image as an input image and the preprocessed second image as a target image.

### CROSS-REFERENCE TO RELATED APPLICATIONS

The present application claims priority to Provisional Application No. 63/620,711, filed Jan. 12, 2024, the contents of which are incorporated herein in their entirety.

### FIELD

The present application relates to medical imaging systems, including positron emission tomography (PET) scanners, Computed Tomography (CT) scanners, etc. Specifically, the application relates to image preprocessing designed to improve the performance of artificial neural networks (ANNs) used in medical imaging systems.

### DESCRIPTION OF THE RELATED ART

The potential applications of artificial neural networks (ANNs) in medical imaging systems have been widely explored in recent years. It is particularly useful to leverage ANN structures and designs that have been developed and optimized for natural images, because there have been large investments in image enhancement technologies in related fields.

In contrast to natural images, the intensities of medical images are not confined to a fixed range and can exhibit significant variations even among images acquired on the same scanner, using the same protocol, for the same application. Furthermore, in some medical imaging applications, the number of different images available for ANN training is much smaller than in traditional ANN-based image enhancement applications. The broad and varied intensity range of medical images can lead to suboptimal learning, especially when the available training dataset is limited.

In an approach addressing the varying-intensity problem, intensity normalization based on the statistical properties, (e.g., mean or standard deviation) of the individual or the entire set of training images has been applied. The intention is to rescale the intensities into a similar range such that the ANN is less distracted and able to allocate its resources for the actual task. However, while this approach focuses the ANN's resources on a narrower intensity range, the ANN still disperses its attention across the entire intensity range, although with narrower intensity bins.

ANNs for image enhancement in medical imaging represent a new technology with unverified applicability and robustness. Therefore, it is desirable to improve the quality of the ANN's output.

### SUMMARY

An embodiment of present application is directed to a method for performing image enhancement using a neural network in a medical imaging system. The method includes acquiring an image of an imaging object, and applying the acquired image to a trained neural network to generate an image-enhanced image of the imaging object. The neural network was trained by receiving a training image pair including a first image and a second image, performing an image intensity preprocessing on the received image pair to generate a preprocessed first image and a preprocessed second image, such that the preprocessed first image has a first intensity range covering a portion of an intensity range of the first image, and the preprocessed second image has a second intensity range covering a portion of an intensity range of the second image, and training the neural network using the preprocessed first image as an input image and the preprocessed second image as a target image.

Another embodiment of the present application is directed to an apparatus for performing image enhancement using a neural network in a medical imaging system. The apparatus includes processing circuitry configured to acquire an image of an imaging object, and apply the acquired image to a trained neural network to generate an image-enhanced image of the imaging object. The neural network was trained by receiving a training image pair including a first image and a second image, performing an image intensity preprocessing on the received image pair to generate a preprocessed first image and a preprocessed second image, such that the preprocessed first image has a first intensity range covering a portion of an intensity range of the first image, and the preprocessed second image has a second intensity range covering a portion of an intensity range of the second image, and training the neural network using the preprocessed first image as an input image and the preprocessed second image as a target image.

A further embodiment of the present application is directed to a method for training a neural network to perform image enhancement in a medical imaging system. The method includes receiving a training image pair including a first image and a second image, performing an image intensity preprocessing on the received image pair to generate a preprocessed first image and a preprocessed second image, such that the preprocessed first image has a first intensity range covering a portion of an intensity range of the first image, and the preprocessed second image has a second intensity range covering a portion of an intensity range of the second image, and using the preprocessed first image as an input image and the preprocessed second image as a target image, training the neural network to obtain a trained neural network.

Note that this summary section does not specify every embodiment and/or incrementally novel aspect of the present application or claimed invention. Instead, the summary only provides a preliminary discussion of different embodiments and corresponding points of novelty. For additional details and/or possible perspectives of the invention and embodiments, the reader is directed to the Detailed Description section and corresponding figures of the present disclosure as further discussed below.

### BRIEF DESCRIPTION OF THE DRAWINGS

The application will be better understood in light of the description, which is given in a non-limiting manner, accompanied by the attached drawings in which:

FIG. 1 shows an exemplary image flow of an approach for mitigating the network distractions caused by the variation in the image intensity range, in accordance with one embodiment of the present application;

FIG. 2 shows a flow chart of an image enhancement processing procedure 200 in accordance with one embodiment of the present application;

FIGS. 3A, 3B, 3C, and 3D exemplarily illustrate how the image visual contrast and intensity distribution change after performing the image preprocessing in accordance with one embodiment of the present application;

FIG. 4 shows a flow chart of an image enhancement processing procedure 400 in accordance with one embodiment of the present application;

FIGS. 5A, 5B, 5C, 5D, 5E, 5F, and 5G exemplarily illustrate how the image visual contrast and intensity distribution change after performing the image preprocessing in accordance with one embodiment of the present application;

FIGS. 6A and 6B illustrates two possible strategies to deploy network(s) on the decomposed sub-images in accordance with one embodiment of the present application;

FIG. 7A shows a perspective view of a positron emission tomography (PET) scanner that can be used with the techniques described herein; and

FIG. 7B shows a schematic view of a PET scanner that can be used with the techniques described herein.

### DETAILED DESCRIPTION

Embodiments or examples for implementing various aspects of the present application may be as set forth in the following sections. Specific examples of components and arrangements are described below to simplify the present application. These are, of course, merely examples and are not intended to be limiting.

The order of discussion of the different steps as described herein is presented for the sake of clarity. In general, these steps can be performed in any suitable order. Additionally, although each of the different features, techniques, configurations, etc. herein may be discussed in different places of this application, it is intended that each of the concepts can be executed independently of each other or in combination with each other. Accordingly, the present application can be embodied and viewed in many different ways.

Furthermore, as used herein, the words “a,” “an,” and the like generally carry a meaning of “one or more,” unless stated otherwise.

Spurious ranges in medical images are commonly observed in nuclear medical imaging (such as tissue infiltration during injection), X-ray/CT imaging (such as intense image artifacts caused by metal), and other modalities. As mentioned above, the broad and varied intensity range of medical images can lead to suboptimal learning, especially when there is a limited training dataset available for artificial neural network (ANN) training.

While normalizing intensity across training images can partly address this problem, the relationship between intensity values and underlying pathological or physiological information can be weakened or even lost during intensity normalization. Moreover, the wide and various intensity range can also hamper the generalization of the network, resulting in inconsistent performance across different images during ANN deployment.

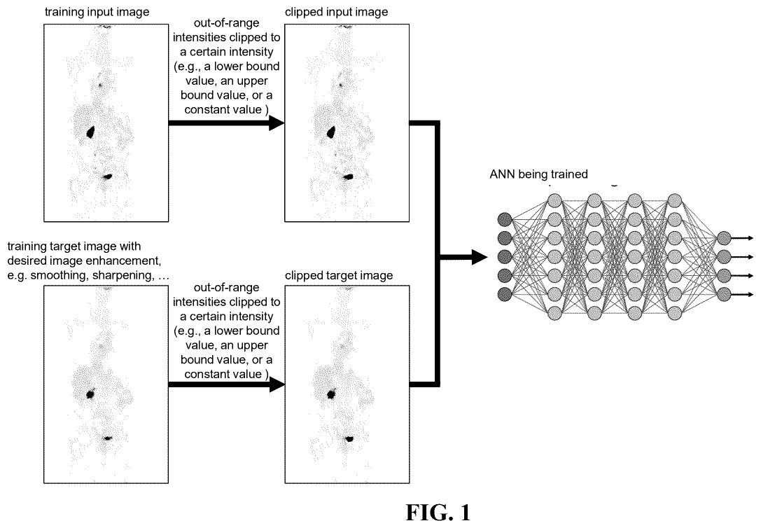

This application provides an alternative approach to mitigate the network distractions caused by the variation in image intensity range without compromising the relationship between intensity values and underlying pathological or physiological information. An exemplary image flow of this approach is illustrated in FIG. 1. The approach trains and deploys the network(s) on images preprocessed to have an effective intensity range covering only segment(s) of the original intensity range. By using images with a relatively narrow and similar intensity range, the network has better training outcome since it is less distracted from its actual task and able to allocate its resources for its actual task.

The details of the provided approach can be found in the following two embodiments.

### Embodiment 1

This embodiment is applicable to scenarios where only intensities within a relatively narrow range, compared to the overall intensity range, show high correlation with the pathology/physiology of interest.

In accordance with Embodiment 1, a lower and/or upper intensity bound can be chosen for preprocessing the training images (including input images and their target images) before feeding them into the network. The selection of the lower/upper bound value(s) is based on a known relationship between the intensity values and the pathology/physiology of interest. Voxel intensities that are lower/higher than the lower/upper bound can be either replaced with the lower/upper intensity bound value(s) or set to a constant. The effective intensity range of the intensity-bounded images is thereby reduced to a fixed and narrow segment of the original intensity range, as determined by the bound value(s). These intensity-bounded images are suitable for training various ANNs. There is no limit on the forms of the network architectures.

For example, a training image set of the ANN can include multiple image pairs, with each image pair including a training input image (e.g., a 3D positron emission tomography (PET) volume) and a corresponding training target image. The target image exhibits a desired image enhancement compared to the input image, including but not limited to, improved image quality, reduced noise, increased smoothness, sharper details, etc. In an example, the training input images and target images are derived from the same data source, such as raw data generated during the same acquisition procedure. For instance, in a training image pair, the input image is a down-sampled, standard-count PET image, while the target image is a high-count PET image. Both images within the same training image pair are clipped using the same lower/upper bound value(s).

Note that the same intensity bound(s) can be applied during network deployment. Voxels with original intensities lower/higher than the lower/upper bound value(s) can be backfilled in the predicted image to restore the original intensity range.

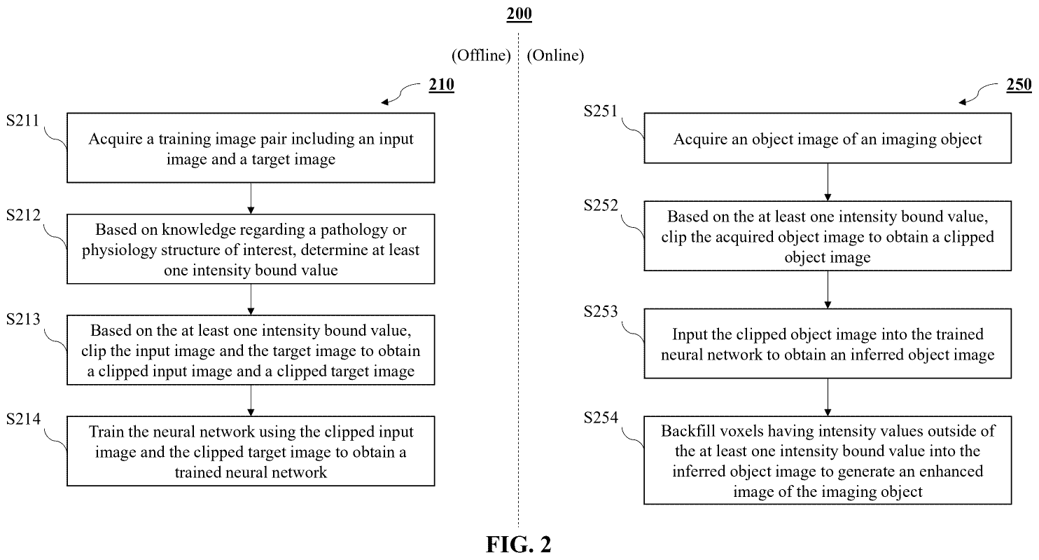

FIG. 2 shows a flow chart of an image enhancement processing procedure 200 according to Embodiment 1. The procedure 200 includes an offline phase 210 for training the neural network and an online phase 250 for applying the trained neural network to image enhancement.

The offline phase 210 starts with step S211 by acquiring a training image pair. The training image pair includes an input image and a corresponding target image. As mentioned above, the input image can be a down-sampled, lower-count PET volume, while the target image can be a high-count PET volume, both derived from the same scan process, for instance. In step S212, at least one intensity bound value (e.g., an intensity upper bound value, an intensity lower bound value, or both an intensity upper and an intensity lower bound values) can be determined based on prior knowledge of a pathology or physiology structure of interest. In step S213, both the training input image and the training target image are clipped based on the at least one intensity bound value to generate a clipped input image and a clipped target image. Voxels with intensity values above the upper bound value can be either set to the intensity upper bound value or another predefined constant value, for example. Alternatively, or additionally, voxels with intensity values below the lower bound value can be either set to the intensity lower bound value or the predefined constant value. In step S214, the clipped input image and the clipped target image are used as an input and a target images to train the neural network.

The trained neural network can then be deployed to enhance images acquired by the medical imaging system from an imaging object. The online phase 250 starts with step S251 by acquiring an object image scanned from the imaging object. In step S252, the acquired object image is clipped based on the at least one intensity bound value to generate a clipped object image. In step S253, the clipped object image is inputted into the trained neural network, and an inferred object image is obtained at the output of the network. In step S254, voxels in the original object image that have intensity values outside of the at least one intensity bound value (i.e., voxels in the original object image that have intensity values lower than the intensity lower bound value, and/or voxels in the original object image that have intensity values higher than the intensity upper bound value) are backfilled into the inferred object image to generate a final enhanced image of the imaging object.

For example, in a scenario of applying an intensity upper bound value, voxels in the original object image that exceed the intensity upper bound value can be extracted and temporarily stored. Once the neural network generates the inferred object image from the clipped object image, these stored voxels can be reinserted into their respective positions, replacing the corresponding voxels in the inferred object image.

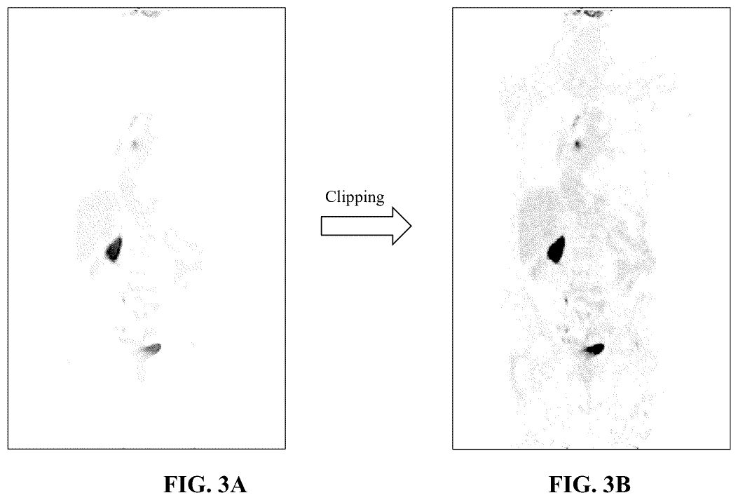

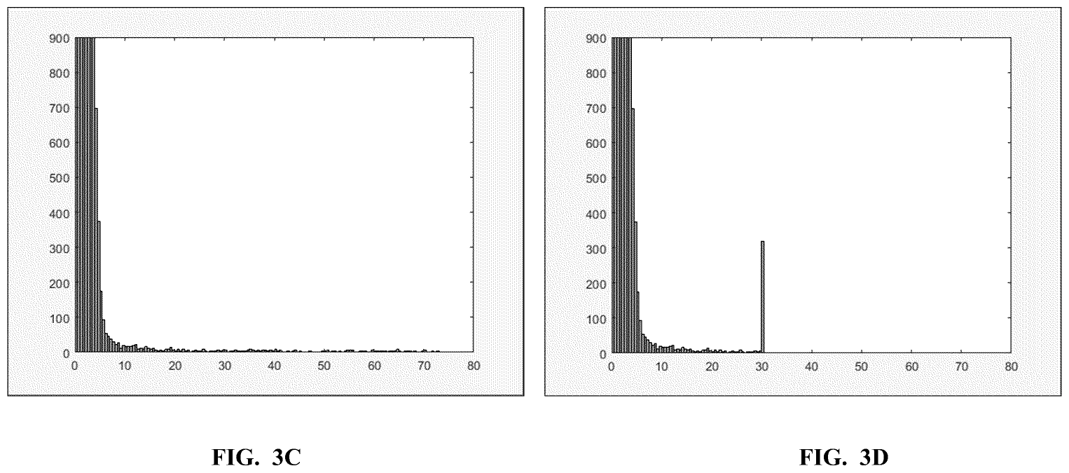

FIGS. 3A, 3B, 3C, and 3D provide an example illustrating how the image visual contrast and intensity distribution change after applying an upper intensity bound of 30, in accordance with Embodiment 1.

FIGS. 3A and 3B show sample coronal slices of an ¹⁸F-FDG whole-body PET study in SUV units, before and after applying an upper intensity bound of 30. This upper intensity bound is chosen based on the common knowledge that the FDG uptake in tissues usually has an SUV value less than 30, while voxels with SUV values higher than 30 are of less interest because they are often observed in the kidneys, ureter, and bladder for the excretion of FDG. Voxel intensities that are higher than the upper bound of 30 are set to 30.

The corresponding intensity distributions are provided in FIGS. 3C and 3D, respectively. The y-axis of the distributions is cropped to facilitate the visualization of the intensity bins with low counts.

### Embodiment 2

This embodiment is applicable to scenarios where all the voxel intensities are of interest.

According to Embodiment 2, the training image (including the training input images and their corresponding target images) can be decomposed into several sub-images before being fed into the network. Each sub-image includes voxels with intensities within a predefined segment of the original intensity range.

The intensity segments can be designed to have an overlap in-between, and thus at least one voxel may be assigned to more than one sub-image. The combination of these intensity segments is equivalent to the overall intensity range.

After the image decomposition, the intensity range of each sub-image is reduced to a fixed and narrow range, determined by the corresponding intensity segment. The sub-images can either be fed into separate networks for independent training, or alternatively, into a single network but different channels for weight-sharing training. A weighting scheme can be performed to blend the outcome from these different networks or channels to assemble the final predicted image in the original intensity range. Similar to Embodiment 1, there is no limit on the forms of the network architectures.

Once again, the same image decomposition can be applied during network deployment.

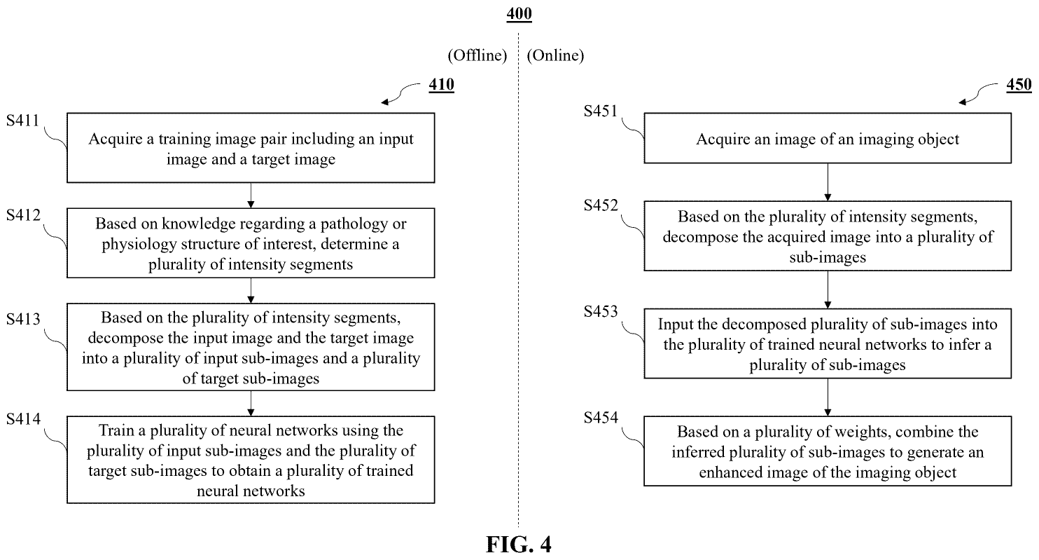

FIG. 4 shows a flow chart of an image enhancement processing procedure 400 according to Embodiment 2. Similar to Embodiment 1, the procedure 400 includes an offline phase 410 for training the neural network and an online phase 450 for applying the trained neural network to image enhancement.

The offline phase 410 starts with step S411 by acquiring a training image pair including an input image and a corresponding target image. In step S412, upon it is decided, based on prior knowledge of a pathology or physiology structure of interest, that the entire intensity range is of interest, a plurality of intensity segments are determined. The combination of these intensity segments collectively covers the entire intensity range of interest. In step S413, both the training input and target image are decomposed based on the determined intensity segments to generate a plurality of sub-images, each corresponding to one of the intensity segments. In step S414, the input sub-images and target sub-images are used to train a set of neural networks, with each network focusing on a specific intensity segment.

Once trained, these neural networks can then be deployed to enhance images acquired by the medical imaging system from an imaging object. The online phase 450 starts with step S451, where an object image is acquired from the imaging object. In step S452, the acquired object image is decomposed based on the determined intensity segments to generate a plurality of object sub-images. In step S453, these object sub-images are inputted into the trained neural networks in a one-by-one manner, generating a plurality of inferred sub-images at the outputs of the networks. In step S454, the inferred sub-images are combined based on a plurality of weights to generate a final enhanced image of the imaging object. For example, the weights can be learned by use of another neural network.

Although FIG. 4 shows the use of separately trained neural networks for different intensity segments, a single network can be trained and deployed. In this approach, the network can have a plurality of channels, with each channel corresponding to a specific intensity segment. The plurality of channels can share one or more network parameters with each other.

The exemplary 18F-FDG whole-body PET image in FIG. 3A can be used again to illustrate the image decomposition process in accordance with Embodiment 2. As an example, the original intensity range is equally divided into three segments with 50% of overlap between adjacent segments.

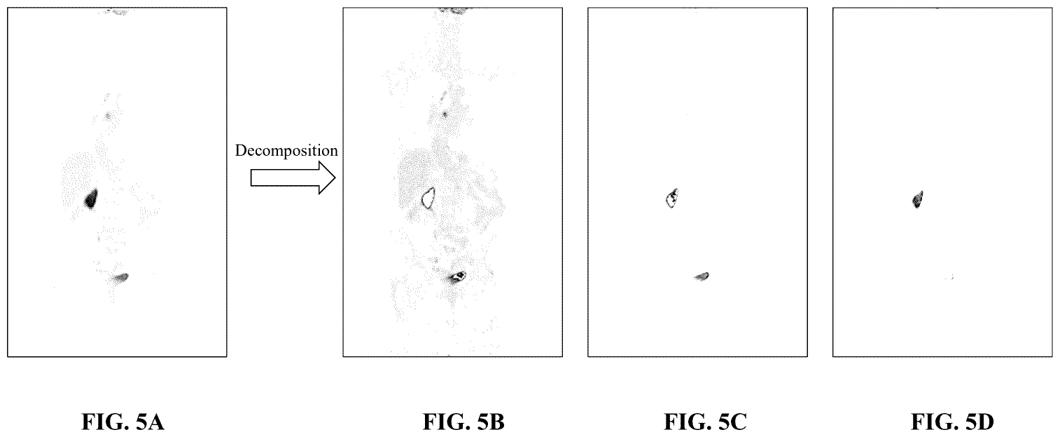

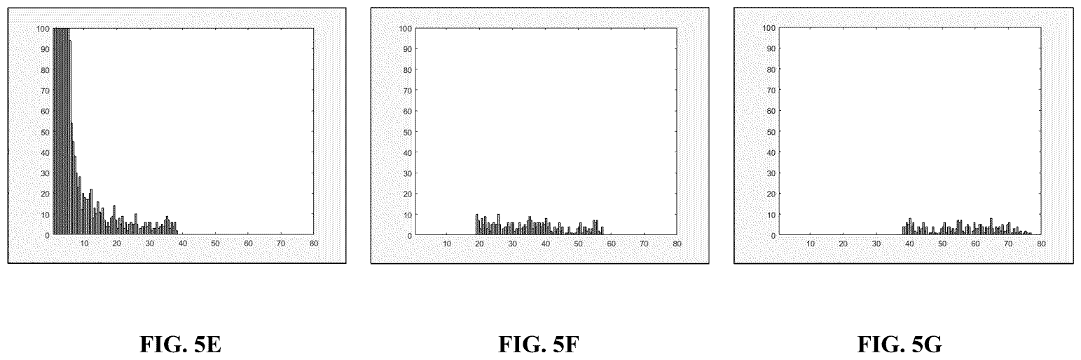

FIGS. 5A and 5B-D show sample coronal slices of the 18F-FDG whole-body PET study in SUV units, before and after the image decomposition. The corresponding intensity distributions are provided in FIGS. 5E, 5F, and 5G, respectively. The y-axis of the distributions is cropped to facilitate the visualization of the intensity bins with low counts.

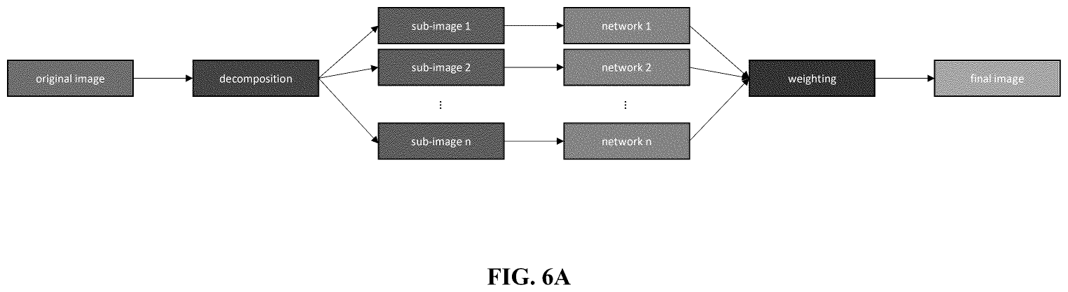

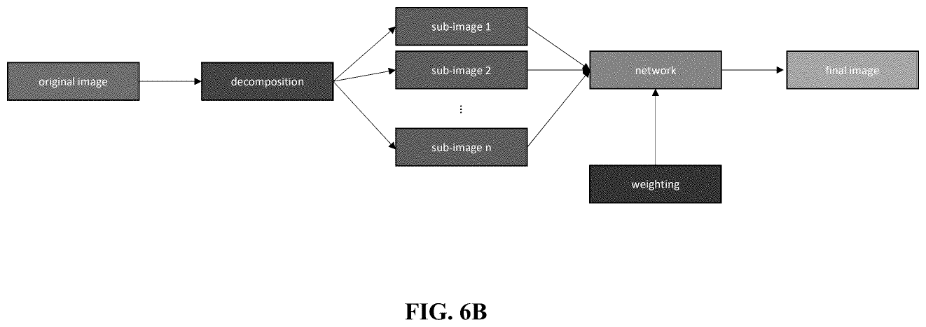

FIGS. 6A and 6B illustrates two possible strategies to deploy network(s) on the decomposed sub-images. Specifically, the sub-images can either be fed into separate networks for independent training (as shown in FIG. 6A) or one single network but different channels for weight-sharing training (as shown in FIG. 6B). A weighting scheme can be implemented to blend the outcome from different networks or channels to assemble the final predicted image in the original intensity range.

The advantages of using images with a relatively narrow and similar intensity range for ANN applications include:
(1) Improved training outcome, as the network is less distracted from the actual task.
(2) Enhanced generalization, leading to more consistent performance during network deployment.

The main difference between the proposed approach and the commonly used intensity normalization approach is the preservation of the relationship between the intensity value and underlying pathological or physiological information. As more information is preserved and passed to the network with less distraction caused by the variation in image intensity range, further improvement in network training and generalization can be expected.

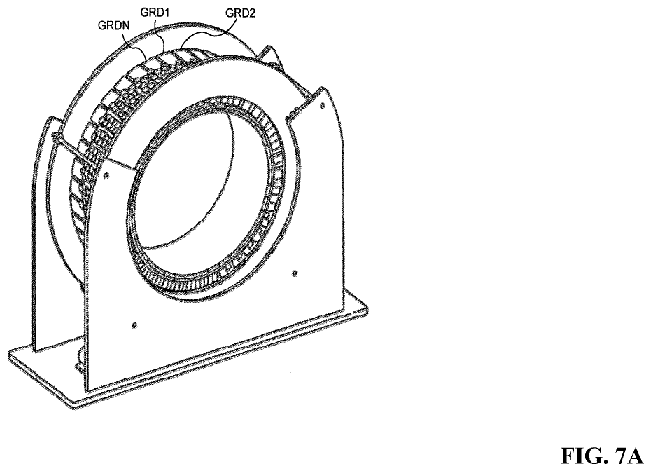

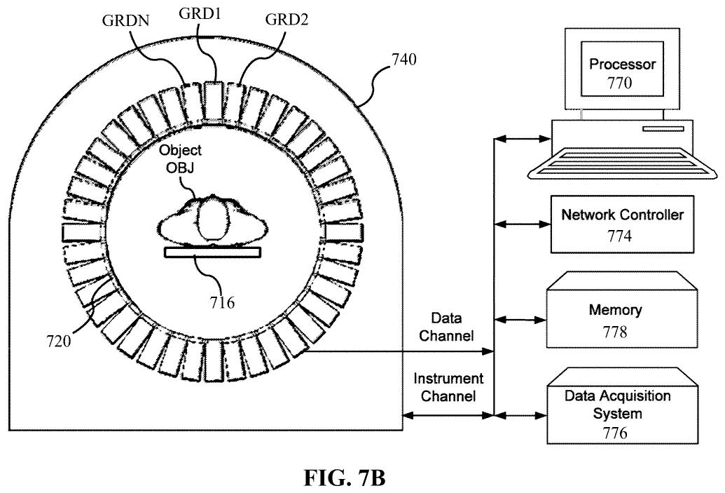

FIGS. 7A and 7B illustrate in implementation in which a medical imaging system includes a PET scanner that can implement the methods described in this application. It is important to note that the image preprocessing approach can be applied to other imaging modalities, including but not limited to X-ray/CT imaging, for example.

In FIG. 7A, the PET scanner includes a plurality of gamma-ray detectors (GRDs) (e.g., GRD1, GRD2, through GRDN) that are each configured as rectangular detector modules. Each GRD can include a two-dimensional array of individual detector crystals, which absorb gamma radiation and emit scintillation photons. The scintillation photons can be detected by a two-dimensional array of photodetectors or photosensors, e.g., photomultiplier tubes (PMTs), silicon photomultipliers (SiPMs), etc. A light guide can be disposed between the array of detector crystals and the photodetectors.

Each photodetector (e.g., PMT or SiPM) can produce an analog signal that indicates when scintillation events occur, and an energy of the gamma-ray producing the detection event. Moreover, the photons emitted from one detector crystal can be detected by more than one photodetector, and, based on the analog signal produced at each photodetector, the detector crystal corresponding to the detection event can be determined using Anger logic and crystal decoding, for example.

FIG. 7B shows one example of the arrangement of the PET scanner, in which the object OBJ to be imaged rests on a table 716 and the GRD modules GRD1 through GRDN are arranged circumferentially around the object OBJ and the table 716. The GRDs can be fixedly connected to a circular component 720 that is fixedly connected to a gantry 740. The gantry 740 houses many parts of the PET scanner. The gantry 740 of the PET scanner also includes an open aperture through which the object OBJ and the table 716 can pass, and gamma-rays emitted in opposite directions from the object OBJ due to an annihilation event can be detected by the GRDs and timing and energy information can be used to determine coincidences for gamma-ray pairs.

In FIG. 7B, circuitry and hardware are also shown for acquiring, storing, processing, and distributing gamma-ray detection data. The circuitry and hardware include: a processor 770, a network controller 774, a memory 778, and a data acquisition system (DAS) 776. The PET scanner also includes a data channel that routes detection measurement results from the GRDs to the DAS 776, the processor 770, the memory 778, and the network controller 774. The data acquisition system 776 can control the acquisition, digitization, and routing of the detection data from the detectors. In one implementation, the DAS 776 controls the movement of the bed 716. The processor 770 performs functions including reconstructing images from the detection data, pre-reconstruction processing of the detection data, and post-reconstruction processing of the image data, as discussed herein.

The processor 770 can be configured to perform various steps of the methods described herein and variations thereof. The processor 770 can include a CPU that can be implemented as discrete logic gates, as an Application Specific Integrated Circuit (ASIC), a Field Programmable Gate Array (FPGA) or other Complex Programmable Logic Device (CPLD). An FPGA or CPLD implementation may be coded in VHDL, Verilog, or any other hardware description language and the code may be stored in an electronic memory directly within the FPGA or CPLD, or as a separate electronic memory. Further, the memory may be non-volatile, such as ROM, EPROM, EEPROM or FLASH memory. The memory can also be volatile, such as static or dynamic RAM, and a processor, such as a microcontroller or microprocessor, may be provided to manage the electronic memory as well as the interaction between the FPGA or CPLD and the memory.

Alternatively, the CPU in the processor 770 can execute a computer program including a set of computer-readable instructions that perform various steps of the described methods, the program being stored in any of the above-described non-transitory electronic memories and/or a hard disk drive, CD, DVD, FLASH drive or any other known storage media. Further, the computer-readable instructions may be provided as a utility application, background daemon, or component of an operating system, or combination thereof, executing in conjunction with a processor, such as a Xeon processor from Intel of America or an Opteron processor from AMD of America and an operating system, such as Microsoft VISTA, UNIX, Solaris, LINUX, Apple, MAC-OS and other operating systems known to those skilled in the art. Further, CPU can be implemented as multiple processors cooperatively working in parallel to perform the instructions.

The memory 778 can be a hard disk drive, CD-ROM drive, DVD drive, FLASH drive, RAM, ROM or any other electronic storage known in the art.

The network controller 774, such as an Intel Ethernet PRO network interface card from Intel Corporation of America, can interface between the various parts of the PET scanner. Additionally, the network controller 774 can also interface with an external network. As can be appreciated, the external network can be a public network, such as the Internet, or a private network such as an LAN or WAN network, or any combination thereof and can also include PSTN or ISDN sub-networks. The external network can also be wired, such as an Ethernet network, or can be wireless such as a cellular network including EDGE, 3G and 4G wireless cellular systems. The wireless network can also be WiFi, Bluetooth, or any other wireless form of communication that is known.

Various techniques have been described as multiple discrete operations to assist in understanding the various embodiments. The order of description should not be construed as to imply that these operations are necessarily order dependent. Indeed, these operations need not be performed in the order of presentation. Operations described may be performed in a different order than the described embodiment. Various additional operations may be performed and/or described operations may be omitted in additional embodiments.

Numerous modifications and variations of the embodiments presented herein are possible in light of the above teachings. It is therefore to be understood that within the scope of the claims, the application may be practiced otherwise than as specifically described herein. The inventions are not limited to the examples that have just been described; it is in particular possible to combine features of the illustrated examples with one another in variants that have not been illustrated.

Embodiments of the present disclosure may also be as set forth in the following parentheticals.

(1) A method for performing image enhancement using a neural network in a medical imaging system, the method comprising: acquiring an image of an imaging object; and applying the acquired image to a trained neural network to generate an image-enhanced image of the imaging object, wherein the neural network was trained by: receiving a training image pair including a first image and a second image, performing an image intensity preprocessing on the received image pair to generate a preprocessed first image and a preprocessed second image, such that the preprocessed first image has a first intensity range covering a portion of an intensity range of the first image, and the preprocessed second image has a second intensity range covering a portion of an intensity range of the second image, and training the neural network using the preprocessed first image as an input image and the preprocessed second image as a target image.

(2) The method of (1), wherein the step of performing the image intensity preprocessing further comprises: based on knowledge with respect to a pathology or physiology structure of interest, determining at least one intensity bound value, clipping, based on the determined at least one intensity bound value, the first image to generate a clipped first image, as the preprocessed first image, and clipping, based on the determined at least one intensity bound value, the second image to generate a clipped second image, as the preprocessed second image.

(3) The method of (2), wherein the determining step further comprises determining a lower bound value, the step of clipping the first image further comprises, when a voxel in the first image has an intensity value lower than the determined lower bound value, setting the intensity value of the voxel to the determined lower bound value or a predefined constant value, and the step of clipping the second image further comprises, when a voxel in the second image has an intensity value lower than the determined lower bound value, setting the intensity value of the voxel to the determined lower bound value or the predefined constant value.

(4) The method of (2), wherein the determining step further comprises determining an upper bound value, the step of clipping the first image further comprises, when a voxel in the first image has an intensity value higher than the determined upper bound value, setting the intensity value of the voxel to the determined upper bound value or a predefined constant value, and the step of clipping the second image further comprises, when a voxel in the second image has an intensity value higher than the determined upper bound value, setting the intensity value of the voxel to the determined upper bound value or the predefined constant value.

(5) The method of (2), wherein the determining step further comprises determining a lower bound value and an upper bound value, the step of clipping the first image further comprises: when a voxel in the first image has an intensity value lower than the determined lower bound value, setting the intensity value of the voxel to the determined lower bound value or a predefined constant value, and when a voxel in the first image has an intensity value higher than the determined upper bound value, setting the intensity value of the voxel to the determined upper bound value or the predefined constant value, and the step of clipping the second image further comprises: when a voxel in the second image has an intensity value lower than the determined lower bound value, setting the intensity value of the voxel to the determined lower bound value or the predefined constant value, and when a voxel in the second image has an intensity value higher than the determined upper bound value, setting the intensity value of the voxel to the determined upper bound value or the predefined constant value.

(6) The method of claim 2, wherein the applying step further comprises: clipping, based on the determined at least one intensity bound value, the acquired image to generate a clipped image, inputting the clipped image into the trained neural network to infer an image at an output of the trained neural network, and for a voxel in the acquired image that has an intensity value outside of the determined at least one intensity bound value, backfilling the voxel in the acquired image into the inferred image to obtain a backfilled image, as the generated image-enhanced image.

(7) The method of (1), wherein the step of performing the image intensity preprocessing further comprises: based on knowledge with respect to a pathology or physiology structure of interest, determining a plurality of intensity segments, decomposing, based on the determined plurality of intensity segments, the first image into a plurality of first sub-images, as the preprocessed first image, and decomposing, based on the determined plurality of intensity segments, the second image into a plurality of second sub-images, as the preprocessed second image.

(8) The method of (7), wherein the neural network includes a plurality of neural networks, and the training step further comprises, for each neural network of the plurality of neural networks, performing training by using one sub-image of the plurality of first sub-images as an input image and a corresponding one sub-image of the plurality of second sub-images as a target image, to obtain a plurality of trained neural networks.

(9) The method of claim 8, wherein the applying step further comprises: decomposing, based on the determined plurality of intensity segments, the acquired image into a plurality of sub-images, inputting the decomposed plurality of sub-image into the plurality of trained neural networks in a one-to-one manner, to infer a plurality of sub-images at a plurality of outputs of the plurality of trained neural networks, and combining, based on a plurality of weights, the inferred plurality of sub-images to obtain a combined image, as the generated image-enhanced image.

(10) The method of (7), wherein the neural network includes a plurality of channels with a network parameter shared thereamong, and the training step further comprises, for each channel of the plurality of channels, performing training by using one sub-image of the plurality of first sub-images as an input image and a corresponding one sub-image of the plurality of second sub-images as a target image.

(11) The method of (10), wherein the applying step further comprises: decomposing, based on the determined plurality of intensity segments, the acquired image into a plurality of sub-images, inputting the decomposed plurality of sub-image into the plurality of channels of the trained neural network in a one-to-one manner, to infer a plurality of sub-images at a plurality of outputs of the plurality of channels of the trained neural network, and combining, based on a plurality of weights, the inferred plurality of sub-images to obtain a combined image, as the generated image-enhanced image.

(12) The method of (7), wherein the determining step further comprises determining the plurality of intensity segments, such that at least two intensity segments of the determined plurality of intensity segments have an overlap between each other.

(13) An apparatus for performing image enhancement using a neural network in a medical imaging system, the apparatus comprising processing circuitry configured to: acquire an image of an imaging object, and apply the acquired image to a trained neural network to generate an image-enhanced image of the imaging object, wherein the neural network was trained by: receiving a training image pair including a first image and a second image, performing an image intensity preprocessing on the received image pair to generate a preprocessed first image and a preprocessed second image, such that the preprocessed first image has a first intensity range covering a portion of an intensity range of the first image, and the preprocessed second image has a second intensity range covering a portion of an intensity range of the second image, and training the neural network using the preprocessed first image as an input image and the preprocessed second image as a target image.

(14) The apparatus of (13), wherein the step of performing the image intensity preprocessing further comprises: based on knowledge with respect to a pathology or physiology structure of interest, determining at least one intensity bound value, clipping, based on the determined at least one intensity bound value, the first image to generate a clipped first image, as the preprocessed first image, and clipping, based on the determined at least one intensity bound value, the second image to generate a clipped second image, as the preprocessed second image.

(15) The apparatus of (14), wherein the determining step further comprises determining a lower bound value, the step of clipping the first image further comprises, when a voxel in the first image has an intensity value lower than the determined lower bound value, setting the intensity value of the voxel to the determined lower bound value or a predefined constant value, and the step of clipping the second image further comprises, when a voxel in the second image has an intensity value lower than the determined lower bound value, setting the intensity value of the voxel to the determined lower bound value or the predefined constant value.

(16) The apparatus of (14), wherein the determining step further comprises determining an upper bound value, the step of clipping the first image further comprises, when a voxel in the first image has an intensity value higher than the determined upper bound value, setting the intensity value of the voxel to the determined upper bound value or a predefined constant value, and the step of clipping the second image further comprises, when a voxel in the second image has an intensity value higher than the determined upper bound value, setting the intensity value of the voxel to the determined upper bound value or the predefined constant value.

(17) The apparatus of (14), wherein the determining step further comprises determining a lower bound value and an upper bound value, the step of clipping the first image further comprises: when a voxel in the first image has an intensity value lower than the determined lower bound value, setting the intensity value of the voxel to the determined lower bound value or a predefined constant value, and when a voxel in the first image has an intensity value higher than the determined upper bound value, setting the intensity value of the voxel to the determined upper bound value or the predefined constant value, and the step of clipping the second image further comprises: when a voxel in the second image has an intensity value lower than the determined lower bound value, setting the intensity value of the voxel to the determined lower bound value or the predefined constant value, and when a voxel in the second image has an intensity value higher than the determined upper bound value, setting the intensity value of the voxel to the determined upper bound value or the predefined constant value.

(18) The apparatus of (14), wherein the applying step further comprises: clipping, based on the determined at least one intensity bound value, the acquired image to generate a clipped image, inputting the clipped image into the trained neural network to infer an image at an output of the trained neural network, and for a voxel in the acquired image that has an intensity value outside of the determined at least one intensity bound value, backfilling the voxel in the acquired image into the inferred image to obtain a backfilled image, as the generated image-enhanced image.

(19) The apparatus of (13), wherein the step of performing the image intensity preprocessing further comprises: based on knowledge with respect to a pathology or physiology structure of interest, determining a plurality of intensity segments, decomposing, based on the determined plurality of intensity segments, the first image into a plurality of first sub-images, as the preprocessed first image, and decomposing, based on the determined plurality of intensity segments, the second image into a plurality of second sub-images, as the preprocessed second image.

(20) A method for training a neural network to perform image enhancement in a medical imaging system, the method comprising: receiving a training image pair including a first image and a second image; performing an image intensity preprocessing on the received image pair to generate a preprocessed first image and a preprocessed second image, such that the preprocessed first image has a first intensity range covering a portion of an intensity range of the first image, and the preprocessed second image has a second intensity range covering a portion of an intensity range of the second image; and using the preprocessed first image as an input image and the preprocessed second image as a target image, training the neural network to obtain a trained neural network.

Numerous modifications and variations of the embodiments presented herein are possible in light of the above teachings. It is therefore to be understood that within the scope of the claims, the disclosure may be practiced otherwise than as specifically described herein.

1. A method for performing image enhancement using a neural network in a medical imaging system, the method comprising:
acquiring an image of an imaging object; and
applying the acquired image to a trained neural network to generate an image-enhanced image of the imaging object, wherein the neural network was trained by:
receiving a training image pair including a first image and a second image,
performing an image intensity preprocessing on the received image pair to generate a preprocessed first image and a preprocessed second image, such that the preprocessed first image has a first intensity range covering a portion of an intensity range of the first image, and the preprocessed second image has a second intensity range covering a portion of an intensity range of the second image, and
training the neural network using the preprocessed first image as an input image and the preprocessed second image as a target image.

2. The method of claim 1, wherein the step of performing the image intensity preprocessing further comprises:
based on knowledge with respect to a pathology or physiology structure of interest, determining at least one intensity bound value,
clipping, based on the determined at least one intensity bound value, the first image to generate a clipped first image, as the preprocessed first image, and
clipping, based on the determined at least one intensity bound value, the second image to generate a clipped second image, as the preprocessed second image.

3. The method of claim 2, wherein the determining step further comprises determining a lower bound value,
the step of clipping the first image further comprises, when a voxel in the first image has an intensity value lower than the determined lower bound value, setting the intensity value of the voxel to the determined lower bound value or a predefined constant value, and
the step of clipping the second image further comprises, when a voxel in the second image has an intensity value lower than the determined lower bound value, setting the intensity value of the voxel to the determined lower bound value or the predefined constant value.

4. The method of claim 2, wherein the determining step further comprises determining an upper bound value,
the step of clipping the first image further comprises, when a voxel in the first image has an intensity value higher than the determined upper bound value, setting the intensity value of the voxel to the determined upper bound value or a predefined constant value, and
the step of clipping the second image further comprises, when a voxel in the second image has an intensity value higher than the determined upper bound value, setting the intensity value of the voxel to the determined upper bound value or the predefined constant value.

5. The method of claim 2, wherein the determining step further comprises determining a lower bound value and an upper bound value,
the step of clipping the first image further comprises:
when a voxel in the first image has an intensity value lower than the determined lower bound value, setting the intensity value of the voxel to the determined lower bound value or a predefined constant value, and
when a voxel in the first image has an intensity value higher than the determined upper bound value, setting the intensity value of the voxel to the determined upper bound value or the predefined constant value, and
the step of clipping the second image further comprises:
when a voxel in the second image has an intensity value lower than the determined lower bound value, setting the intensity value of the voxel to the determined lower bound value or the predefined constant value, and
when a voxel in the second image has an intensity value higher than the determined upper bound value, setting the intensity value of the voxel to the determined upper bound value or the predefined constant value.

6. The method of claim 2, wherein the applying step further comprises:
clipping, based on the determined at least one intensity bound value, the acquired image to generate a clipped image,
inputting the clipped image into the trained neural network to infer an image at an output of the trained neural network, and
for a voxel in the acquired image that has an intensity value outside of the determined at least one intensity bound value, backfilling the voxel in the acquired image into the inferred image to obtain a backfilled image, as the generated image-enhanced image.

7. The method of claim 1, wherein the step of performing the image intensity preprocessing further comprises:
based on knowledge with respect to a pathology or physiology structure of interest, determining a plurality of intensity segments,
decomposing, based on the determined plurality of intensity segments, the first image into a plurality of first sub-images, as the preprocessed first image, and
decomposing, based on the determined plurality of intensity segments, the second image into a plurality of second sub-images, as the preprocessed second image.

8. The method of claim 7, wherein the neural network includes a plurality of neural networks, and the training step further comprises, for each neural network of the plurality of neural networks, performing training by using one sub-image of the plurality of first sub-images as an input image and a corresponding one sub-image of the plurality of second sub-images as a target image, to obtain a plurality of trained neural networks.

9. The method of claim 8, wherein the applying step further comprises:
decomposing, based on the determined plurality of intensity segments, the acquired image into a plurality of sub-images,
inputting the decomposed plurality of sub-image into the plurality of trained neural networks in a one-to-one manner, to infer a plurality of sub-images at a plurality of outputs of the plurality of trained neural networks, and
combining, based on a plurality of weights, the inferred plurality of sub-images to obtain a combined image, as the generated image-enhanced image.

10. The method of claim 7, wherein the neural network includes a plurality of channels with a network parameter shared thereamong, and the training step further comprises, for each channel of the plurality of channels, performing training by using one sub-image of the plurality of first sub-images as an input image and a corresponding one sub-image of the plurality of second sub-images as a target image.

11. The method of claim 10, wherein the applying step further comprises:
decomposing, based on the determined plurality of intensity segments, the acquired image into a plurality of sub-images,
inputting the decomposed plurality of sub-image into the plurality of channels of the trained neural network in a one-to-one manner, to infer a plurality of sub-images at a plurality of outputs of the plurality of channels of the trained neural network, and
combining, based on a plurality of weights, the inferred plurality of sub-images to obtain a combined image, as the generated image-enhanced image.

12. The method of claim 7, wherein the determining step further comprises determining the plurality of intensity segments, such that at least two intensity segments of the determined plurality of intensity segments have an overlap between each other.

13. An apparatus for performing image enhancement using a neural network in a medical imaging system, the apparatus comprising processing circuitry configured to:
acquire an image of an imaging object, and
apply the acquired image to a trained neural network to generate an image-enhanced image of the imaging object, wherein the neural network was trained by:
receiving a training image pair including a first image and a second image,
performing an image intensity preprocessing on the received image pair to generate a preprocessed first image and a preprocessed second image, such that the preprocessed first image has a first intensity range covering a portion of an intensity range of the first image, and the preprocessed second image has a second intensity range covering a portion of an intensity range of the second image, and
training the neural network using the preprocessed first image as an input image and the preprocessed second image as a target image.

14. The apparatus of claim 13, wherein the step of performing the image intensity preprocessing further comprises:
based on knowledge with respect to a pathology or physiology structure of interest, determining at least one intensity bound value,
clipping, based on the determined at least one intensity bound value, the first image to generate a clipped first image, as the preprocessed first image, and
clipping, based on the determined at least one intensity bound value, the second image to generate a clipped second image, as the preprocessed second image.

15. The apparatus of claim 14, wherein the determining step further comprises determining a lower bound value,
the step of clipping the first image further comprises, when a voxel in the first image has an intensity value lower than the determined lower bound value, setting the intensity value of the voxel to the determined lower bound value or a predefined constant value, and
the step of clipping the second image further comprises, when a voxel in the second image has an intensity value lower than the determined lower bound value, setting the intensity value of the voxel to the determined lower bound value or the predefined constant value.

16. The apparatus of claim 14, wherein the determining step further comprises determining an upper bound value,
the step of clipping the first image further comprises, when a voxel in the first image has an intensity value higher than the determined upper bound value, setting the intensity value of the voxel to the determined upper bound value or a predefined constant value, and
the step of clipping the second image further comprises, when a voxel in the second image has an intensity value higher than the determined upper bound value, setting the intensity value of the voxel to the determined upper bound value or the predefined constant value.

17. The apparatus of claim 14, wherein the determining step further comprises determining a lower bound value and an upper bound value,
the step of clipping the first image further comprises:
when a voxel in the first image has an intensity value lower than the determined lower bound value, setting the intensity value of the voxel to the determined lower bound value or a predefined constant value, and
when a voxel in the first image has an intensity value higher than the determined upper bound value, setting the intensity value of the voxel to the determined upper bound value or the predefined constant value, and
the step of clipping the second image further comprises:
when a voxel in the second image has an intensity value lower than the determined lower bound value, setting the intensity value of the voxel to the determined lower bound value or the predefined constant value, and
when a voxel in the second image has an intensity value higher than the determined upper bound value, setting the intensity value of the voxel to the determined upper bound value or the predefined constant value.

18. The apparatus of claim 14, wherein the applying step further comprises:
clipping, based on the determined at least one intensity bound value, the acquired image to generate a clipped image,
inputting the clipped image into the trained neural network to infer an image at an output of the trained neural network, and
for a voxel in the acquired image that has an intensity value outside of the determined at least one intensity bound value, backfilling the voxel in the acquired image into the inferred image to obtain a backfilled image, as the generated image-enhanced image.

19. The apparatus of claim 13, wherein the step of performing the image intensity preprocessing further comprises:
based on knowledge with respect to a pathology or physiology structure of interest, determining a plurality of intensity segments,
decomposing, based on the determined plurality of intensity segments, the first image into a plurality of first sub-images, as the preprocessed first image, and
decomposing, based on the determined plurality of intensity segments, the second image into a plurality of second sub-images, as the preprocessed second image.

20. A method for training a neural network to perform image enhancement in a medical imaging system, the method comprising:
receiving a training image pair including a first image and a second image;
performing an image intensity preprocessing on the received image pair to generate a preprocessed first image and a preprocessed second image, such that the preprocessed first image has a first intensity range covering a portion of an intensity range of the first image, and the preprocessed second image has a second intensity range covering a portion of an intensity range of the second image; and
using the preprocessed first image as an input image and the preprocessed second image as a target image, training the neural network to obtain a trained neural network.
